SEQUENCE LISTING (5 sequences)

SEQ ID NO: 1 - DNA, 2204 bp, Homo sapiens
  CDS (3)..(908)
ccgccctcgtgcccaaggcaggactggccaagcccccagctgctgccaaatccagcccttccctcgcctcttcgtcctcgtcctcgtcctccgcggtggccggtggggccccggagcagcaggccctcctgaggaggggcaagaggcacctgcagggggacggtctcagcagcttcgactccagaggcagccggcccaccacagagactgagttcatcgcctgggggcccacgggggacgaggaggccctggagtccaacacatttccgggcgtttacggccccaccacggtctccatcctacaaacacggaagacaactgtggccgccaccaccaccaccaccaccacggccacccccatgacgctgcagactaaggggttcaccgagtccttggatccccggagaaggatcccaggtggggttagcacaacggagccttccaccagtcccagcaacaacggggaagtcacccagcccccaaggattctgggggaggcctcaggtctggctgtccatcagatcatcaccatcaccgtctccctcatcatggtcatagctgctctcatcacaactcttgtcttaaaaaattgctgtgcccaaagcgggaacactcgtcggaacagccaccagcggaagaccaaccagcaggaggagagctgccagaacctcacggacttcccctcggcccgggtgcccagcagcctggacatattcacggcctataacgagaccctgcagtgttctcacgagtgcgtcagggcatctgtgcccgtgtacaccgatgagacgctgcactcgacgacgggggagtacaaatccacatttaatggaaaccgaccctcctcttctgatcggcatcttattcctgtggccttcgtgtctgagaaatggtttgaaatctcctgctgactggccgaagtcttttttacctcctgggggcagggcagacgccgtgtgtctgtttcacggattccgttggtgaacctgtaaaaacaaaacaaacaaaacaaaacaaaaaagacaaaacctaaaactgagctatctaagggggagggtccccgcacctaccacttctgtttgccggtgggaaactcacagagcaggacgctctaggccaaatctatttttgtaaaaatgctcatgcctatgggtgactgccttctcccagagttttctttggagaacagaaagaagaaaggaaagaaaggaaccagaggcagagagacgaggatacccagcgaaagggacgggaggaagcatccgaaacctaggattcgtcctacgattctgaacctgtgccaataataccattatgtgccatgtactgacccgaaaggctcggccacagagccggggcccagcgaatcacgcagagaaatcttacagaaaacaggggtgggaatctcttccgatagagtcgctatttctggttaatatacatatataaatatataaatacaaacacacacacacactttttttgtactgtagcaatttttgaagatcttaaatgttcctttttaaaaaaaagaattgtgttataggttacaaaatctgatttatttaacatgcttagtatgagcagaataaaccagtgttttctactttggcaactcacgtcacacacatattacacacatgtgcgcatacacacacacaatacacatatatgcatatagacgcatctattggaaatgcagttccacaggtgagcatgttctttctggtgacctggtattccatcaccattcaccccaggggacagcctcgaccgagacaaggaggcccttaaatgacagcctgcatttgctagacggttggtgagtggcatcaaatgtgtgacttactatcttgggccagaactaagaatgccaaggttttatatatgtgtgtatatatatatatatatatatatatatatgtttgtgtgtgtatatatatatatatatatatatgtttgtgtgtgtatatatatgtttgtgtatatatatacacatatgcatacatatgattttttttttttcatttaagtgttggaagatgctacctaacagccacgttcacatttacgtagctggttgcttacaaacgggcctgagcccctggttgggtgggtggtggattcttggacgtgtgtgtcatacaagcatagactggattaaagaagttttccagttccaaaaattaaaggaatatatcctt

SEQ ID NO: 2 - protein, 302 aa, Homo sapiens
ALVPKAGLAKPPAAAKSSPSLASSSSSSSSAVAGGAPEQQALLRRGKRHLQGDGLSSFDSRGSRPTTETEFIAWGPTGDEEALESNTFPGVYGPTTVSILQTRKTTVAATTTTTTTATPMTLQTKGFTESLDPRRRIPGGVSTTEPSTSPSNNGEVTQPPRILGEASGLAVHQIITITVSLIMVIAALITTLVLKNCCAQSGNTRRNSHQRKTNQQEESCQNLTDFPSARVPSSLDIFTAYNETLQCSHECVRASVPVYTDETLHSTTGEYKSTFNGNRPSSSDRHLIPVAFVSEKWFEISC

SEQ ID NO: 3 - DNA, 2288 bp, Homo sapiens
  CDS (3)..(992)
cccggccgccccgagtggagcggatccacgggcagatgcagatgcctcgagccagacgggcccacaggccccgggaccaggcggccgccctcgtgcccaaggcaggactggccaagcccccagctgctgccaaatccagcccttccctcgcctcttcgtcctcgtcctcgtcctccgcggtggccggtggggccccggagcagcaggccctcctgaggaggggcaagaggcacctgcagggggacggtctcagcagcttcgactccagaggcagccggcccaccacagagactgagttcatcgcctgggggcccacgggggacgaggaggccctggagtccaacacatttccgggcgtttacggccccaccacggtctccatcctacaaacacggaagacaactgtggccgccaccaccaccaccaccaccacggccacccccatgacgctgcagactaaggggttcaccgagtccttggatccccggagaaggatcccaggtggggttagcacaacggagccttccaccagtcccagcaacaacggggaagtcacccagcccccaaggattctgggggaggcctcaggtctggctgtccatcagatcatcaccatcaccgtctccctcatcatggtcatagctgctctcatcacaactcttgtcttaaaaaattgctgtgcccaaagcgggaacactcgtcggaacagccaccagcggaagaccaaccagcaggaggagagctgccagaacctcacggacttcccctcggcccgggtgcccagcagcctggacatattcacggcctataacgagaccctgcagtgttctcacgagtgcgtcagggcatctgtgcccgtgtacaccgatgagacgctgcactcgacgacgggggagtacaaatccacatttaatggaaaccgaccctcctcttctgatcggcatcttattcctgtggccttcgtgtctgagaaatggtttgaaatctcctgctgactggccgaagtcttttttacctcctgggggcagggcagacgccgtgtgtctgtttcacggattccgttggtgaacctgtaaaaacaaaacaaacaaaacaaaacaaaaaagacaaaacctaaaactgagctatctaagggggagggtccccgcacctaccacttctgtttgccggtgggaaactcacagagcaggacgctctaggccaaatctatttttgtaaaaatgctcatgcctatgggtgactgccttctcccagagttttctttggagaacagaaagaagaaaggaaagaaaggaaccagaggcagagagacgaggatacccagcgaaagggacgggaggaagcatccgaaacctaggattcgtcctacgattctgaacctgtgccaataataccattatgtgccatgtactgacccgaaaggctcggccacagagccggggcccagcgaatcacgcagagaaatcttacagaaaacaggggtgggaatctcttccgatagagtcgctatttctggttaatatacatatataaatatataaatacaaacacacacacacactttttttgtactgtagcaatttttgaagatcttaaatgttcctttttaaaaaaaagaattgtgttataggttacaaaatctgatttatttaacatgcttagtatgagcagaataaaccagtgttttctactttggcaactcacgtcacacacatattacacacatgtgcgcatacacacacacaatacacatatatgcatatagacgcatctattggaaatgcagttccacaggtgagcatgttctttctggtgacctggtattccatcaccattcaccccaggggacagcctcgaccgagacaaggaggcccttaaatgacagcctgcatttgctagacggttggtgagtggcatcaaatgtgtgacttactatcttgggccagaactaagaatgccaaggttttatatatgtgtgtatatatatatatatatatatatatatatgtttgtgtgtgtatatatatatatatatatatatgtttgtgtgtgtatatatatgtttgtgtatatatatacacatatgcatacatatgattttttttttttcatttaagtgttggaagatgctacctaacagccacgttcacatttacgtagctggttgcttacaaacgggcctgagcccctggttgggtgggtggtggattcttggacgtgtgtgtcatacaagcatagactggattaaagaagttttccagttccaaaaattaaaggaatatatcctt

SEQ ID NO: 4 - protein, 330 aa, Homo sapiens
RPPRVERIHGQMQMPRARRAHRPRDQAAALVPKAGLAKPPAAAKSSPSLASSSSSSSSAVAGGAPEQQALLRRGKRHLQGDGLSSFDSRGSRPTTETEFIAWGPTGDEEALESNTFPGVYGPTTVSILQTRKTTVAATTTTTTTATPMTLQTKGFTESLDPRRRIPGGVSTTEPSTSPSNNGEVTQPPRILGEASGLAVHQIITITVSLIMVIAALITTLVLKNCCAQSGNTRRNSHQRKTNQQEESCQNLTDFPSARVPSSLDIFTAYNETLQCSHECVRASVPVYTDETLHSTTGEYKSTFNGNRPSSSDRHLIPVAFVSEKWFEISC

SEQ ID NO: 5 - DNA, 218 bp, Homo sapiens
gcggttgtccggaatgccagtggctcctgggcagatgtgcaccccagattcagcctttgtgatagattccaacacgttctggcctcagaccacctttgtggtggggccagactgctctgggcaaagtgaagctggcctttatgctccaaggaagggggcctcgagagcaggcctgcattggctctcggactaattcgcgatcatctttcatacagcag

(19) United States
(12) Patent Application Publication
(10) Pub. No.: US 2008/0187527 A1
(43) Pub. Date: Aug. 7, 2008

(54) Novel endometriosis-associated gene

(76) Inventors: Anna Starzinski-Powitz, Frankfurt (DE); Silvia Zickenheiner, Bad Soden (DE); Heike Handrow-Metzmacher, Frankfurt/Main (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/609,691

(22) Filed: Jul. 1, 2003

Related U.S. Application Data
(63) Continuation-in-part of application No. 09/725,311, filed on Nov. 29, 2000, now Pat. No. 6,586,569.

(30) Foreign Application Priority Data
May 29, 1998 (DE) 198 24 230.1

(51) Int. Cl. A61K 39/00; C07H 21/04; C07K 16/00; C12N 15/00; A61K 38/00; C12N 5/00; A61K 31/70

(52) U.S. Cl. 424/130.1; 536/23.1; 536/23.5; 530/350; 435/320.1; 435/325; 530/387.1; 514/44; 514/12

(57) ABSTRACT

The invention relates to a gene associated with invasive processes, e.g. endometriosis, to a polypeptide coded by said gene, to an antibody directed against the polypeptide, and to the pharmaceutical application of the nucleic acid, the polypeptide and the antibody.

This application is a continuation-in-part of U.S. Ser. No. 09/725,311, filed Nov. 29, 2000.

The present invention relates to a gene associated with invasive processes, for example endometriosis, to a polypeptide encoded by it, to an antibody directed against the polypeptide, and to the pharmaceutical application of the nucleic acid, the polypeptide and the antibody.

Endometriosis is the second most common disease in women and is defined as the occurrence of endometrial cells outside the womb. Endometriosis affects about one in five women of reproductive age, and as many as one in two women with fertility problems.

In normal circumstances the endometrium is only found in the womb. In endometriosis, tissue with a histological appearance resembling the endometrium is found outside the womb, for example externally on the womb, on the intestine or even in the pancreas or the lung. Although these endometriotic foci are located outside the womb, they also bleed during menstruation, thus they are influenced by hormones of the female cycle. Since endometriotic foci like the endometrium go through volume changes during the cycle, these changes may cause pain depending on location. Moreover, the body reacts to endometriotic cells with an inflammatory response which again causes pain. Furthermore, inflammation leads to adhesions in the area of the ovaries and fallopian tubes and, as a result of these, is responsible for a so-called mechanical sterility of affected women. Apparently however, in endometriosis messengers are released as well (e.g. cytokines, prostaglandins) which can reduce the fertility of affected women even in the absence of adhesions.

In view of their pathobiological properties, endometriotic cells could be classified as being between normal cells and tumor cells: on the one hand they show no neoplastic behavior, on the other hand, however, they are, like metastasizing tumor cells, capable of moving across organ boundaries in the organism and of growing into other organs, i.e. they show invasive behavior. For this reason endometriotic cells are defined as “benign tumor cells” in the literature, although up until now no tumor-specific mutations in proto-oncogenes have been found in cells of this type.

Since the pathogenesis of endometriosis is still not clarified completely, there are as yet no effective options for the therapy or prevention of endometriosis-associated diseases.

It was the object of the invention to identify novel genes which play a role in invasive processes and which may be associated with the pathophysiological phenotype of endometriosis.

This object is achieved according to the invention by identifying, cloning and characterizing a gene which is called an endometriosis-associated gene and which codes for a polypeptide. This gene sequence was discovered with the aid of differential display RT-PCR (Liang and Pardee, Science 257 (1992), 967-971). For this, invasive and noninvasive variants of an endometriotic cell line were compared with each other. In the process a cDNA sequence was found which is specific for the invasive variant of endometriotic cells. An associated RNA of 4 kb in length was found. A corresponding cDNA isolated from a cDNA phage bank has an open reading frame (ORF) of 302 amino acids.

The present invention relates to a nucleic acid which comprises

(a) the nucleotide sequences depicted in SEQ ID NO. 1, 3 or/and 5, a combination or a protein-encoding segment thereof,

(b) a nucleotide sequence corresponding to the sequence in (a) within the scope of the degeneracy of the genetic code or

(c) a nucleotide sequence hybridizing with the sequences in (a) and/or (b) under stringent conditions.

The nucleic acids preferably code for a polypeptide associated with invasive processes or a segment thereof.

The following nucleotide sequences have been deposited in the EMBL EST database with the following accession numbers: Z98886, Ac003017, AL023586, Aa52993, Aa452856. These sequences do not represent nucleic acids according to the invention. The first two of these sequences are DNAs which were isolated from human brain and show over 90% identical bases to SEQ. ID NO. 1 in the segments from nucleotide 970 to about 2000 and from 760 to about 1450, respectively, or in the segments from nucleotide 1054 to 2084 and from 844 to about 1534 in relation to SEQ ID NO. 3 which has 84 additional bases at the 5′ end. AL023586 is also a human sequence which is very similar to Z98885 and also has homology with SEQ ID NO. 1 in the region from 970 to about 2000.

Sequences Aa452993 and Aa452856 originate from mouse embryos and show base identity with the nucleotides (nt) from about 1060 to about 1450 and from about 24 to 440, respectively, of SEQ. ID NO. 1, or from about 1144 to about 1534 and from about 108 to about 524, respectively, according to the nucleotide positions in SEQ. ID NO. 3. Up until now no reading frame or function has been assigned to any of these 4 sequences.

The nucleotide sequence depicted in SEQ. ID NO. 1 contains an open reading frame which corresponds to a polypeptide having a length of 302 amino acids. This polypeptide is indicated in the amino acid sequence depicted SEQ. ID NO. 2. SEQ. ID NO. 3 shows a nucleotide sequence as in SEQ. ID NO. 1, but it has 84 additional nucleotides at the 5′ end. As a result, the positions of the nucleotides corresponding to each other shift by 84 nucleotides in each case. The polypeptide encoded by SEQ. ID NO. 3 therefore has 28 additional amino acids at the. N terminus and is depicted in SEQ. ID NO. 4 with its total of 330 amino acids. SEQ. ID NO. 2 and 4 depict a C-terminal segment of the native polypeptide.

For illustration purposes reference is made to FIG. 1 which shows a diagrammatic representation of the cDNA of the endometriosis-associated gene according to the invention. Five exons, E1 to E5, and the position of fragment 1 (394 nt) used as a probe in DDRT-PCR are shown. The positions of the PCR primers (see example 4, table 1) used for RT-PCR are also shown.

Not shown in FIG. 1 is a further exon 4a whose nucleotide sequence is shown in SEQ. ID NO. 5. This exon 4a may be present. If it is present, it is found between exon 4 and exon 5. This corresponds to the position between nt1054 and nt1055 in SEQ. ID NO. 3. A combination of the sequences SEQ. ID NO. 1/3 with SEQ. ID NO. 5 is accordingly, for example, a sequence which contains the sequence of the exon 4a at said position.

Besides the nucleotide sequences shown in SEQ. ID NO. 1, 3 and 5 and combinations thereof such as the sequence of SEQ. ID NO. 3, which has the sequence of SEQ. ID NO. 5 between nt1054 and 1055 and to a nucleotide sequences which corresponds to the sequences within the scope of the degeneracy of the genetic code, the present invention also includes nucleotide sequences which hybridize with one of the sequences mentioned before.

The term “hybridization” according to the present invention is used by Sambrook et al. (Molecular Cloning, A Laboratory Manual, Cold Spring Harbor Laboratory Press (1989), 1.101-1.104). Preferably a hybridization is called stringent if a positive hybridization signal is still observed after washing for one hour with 1×SSC and 0.1% SDS at 50° C., preferably at 55° C., particularly preferably at 62° C. and most preferably at 68° C., in particular for 1 h in 0.2×SSC and 0.1% SDS at 55° C., preferably at 55° C., particularly preferably at 62° C. and most preferably at 68° C. A nucleotide sequence hybridizing under these washing conditions with one or more of the nucleotide sequences depicted in SEQ ID NO. 1, 3 and 5, or with a nucleic sequence corresponding to these sequences within the scope of the degeneracy of the genetic code, is a nucleotide sequence according to the invention.

The nucleotide sequence according to the invention is preferably a DNA. However, it can also include an RNA or a nucleic acid analog such as a peptidic nucleic acid, for example. Particularly preferably the nucleic acid according to the invention includes a protein-encoding segment of the nucleotide sequences depicted in SEQ ID NO. 1, 3 and/or 5 or a sequence having a homology of more than 80%, preferably more than 90% and particularly preferably more than 95% to the nucleotide sequences depicted in SEQ ID NO. 1, 3 or 5 or a segment of preferably at least 20 nucleotides (nt) and particularly preferably at least 50 nt thereof. The same also holds for nucleic acids which have, as described above, the sequence of SEQ. ID NO. 5 in addition to those of SEQ ID NO. 1 or 3. The homology is given in percent identical positions when two nucleic acids (or peptide chains) are compared, where a 100% homology means complete identity of the compared chain molecules (Herder: Lexikon der Biochemie und Molekularbiologie [Dictionary of biochemistry and molecular biology], Spektrum Akademischer Verlag 1995).

Nucleic acids according to the invention are preferably obtainable from mammals and in particular from humans. They may be isolated according to known techniques by using short segments of the nucleotide sequences shown in SEQ. ID NO. 1, 3 or/and 5 as hybridization probes and/or as amplification primers. Furthermore, the nucleic acids according to the invention may also be prepared by chemical synthesis, it being possible to employ modified nucleotide building blocks, for example 2′-O-alkylated nucleotide building blocks, where appropriate, instead of conventional nucleotide building blocks.

The nucleic acids according to the invention or segments thereof may therefore be used for preparing primers and probes which preferably contain markers or labeling groups. Preference is also given to intron-bridging oligonucleotide primers which are particularly suitable for identifying different mRNA species.

The present invention further relates to polypeptides encoded by the nucleic acids defined as above. These polypeptides preferably comprise

(a) the amino acid sequence depicted in SEQ ID NO. 2 or 4 or

(b) a homology of more than 70%, preferably of more than 80% and particularly preferably of more than 90% to the amino acid sequence according to (a).

Besides the polypeptides depicted in SEQ ID NO. 2 or 4, the invention also relates to muteins, variants and fragments thereof. These are sequences which differ from the amino acid sequences depicted in SEQ ID NO. 2 or 4 by substitution, deletion and/or insertion of single amino acids or of short amino acid segments.

The term “variant” includes both naturally occurring allelic variations or splicing variations of the endometriotic protein, and proteins generated by recombinant DNA technology (in particular in vitro mutagenesis with the aid of chemically synthesised oligonucleotides) which correspond substantially to the proteins depicted in SEQ ID NO. 2 or 4 with respect to their biological and/or immunological activity. This term also includes chemically modified polypeptides. Polypeptides which are modified at the termini and/or in the reactive amino acid side groups by acylation, for example acetylation or amidation belong to this group. Polypeptide fragments (peptides) representing a segment of at least 10 amino acids of the amino acid sequence shown in SEQ ID NO. 2 or 4 also belong to the amino acid sequences according to the invention.

The present invention further relates to a vector containing at least one copy of a nucleic acid according to the invention. This vector may be any prokaryotic or eukaryotic vector on which the DNA sequence according to the invention, preferably linked to expression signals such as promoter, operator, enhancer etc., is located. Examples of prokaryotic vectors are chromosomal vectors such as bacteriophages and extrachromosomal vectors such as plasmids, with circular plasmid vectors being particularly preferred. Suitable prokaryotic vectors are described, for example, in Sambrook et al., supra, Chapters 1-4. Particularly preferred is the vector according to the invention, a eukaryotic vector, e.g. a yeast vector, or a vector suitable for higher cells, e.g. plasmid vector, viral vector or plant vector. Vectors of this type are well known to the skilled worker in the field of molecular biology so that there is no need for further explanation here. In particular, reference is made in this connection to Sambrook et al., supra, Chapter 16.

The invention also relates to a vector which contains a segment of at least 21 nucleotides in length of the sequences depicted in SEQ ID NO. 1, 3 or/and 5 or a combination thereof. Preferably this segment has a nucleotide sequence which originates from the protein-encoding region of said sequences or from a region essential for the expression of the protein or polypeptide. These nucleic acids are particularly suitable for preparing therapeutically employable antisense nucleic acids preferably of up to 50 nucleotides in length.

The present invention further relates to a cell transformed with a nucleic acid according to the invention or a vector according to the invention. The cell can be both a eukaryotic and a prokaryotic cell. Methods for transforming cells with nucleic acids are general prior art and therefore need no further explanation. Examples of preferred cells are eukaryotic cells, in particular animal and particularly preferably mammalian cells.

The present invention further relates to an antibody or a fragment of such an antibody against the polypeptide(s) encoded by the endometriosis gene or against variants thereof. Antibodies of this type are particularly preferably directed against complete polypeptides encoded by it or against a peptide sequence corresponding to amino acids 1-330 of the amino acid sequence depicted in SEQ ID NO. 4.

Identification, isolation and expression of a gene according to the invention which is specifically associated with invasive processes and in particular with endometriosis provide the requirements for diagnosis, therapy and prevention of diseases based on those disorders mentioned above.

It becomes possible with the aid of a polypeptide according to the invention or fragments of this polypeptide as immunogen to prepare antibodies against those polypeptides. Preparation of antibodies may be carried out in the usual way by immunizing experimental animals with the complete polypeptide or fragments thereof and subsequently obtaining the resulting polyclonal antisera. According to the method of Köhler and Milstein and its developments monoclonal antibodies can be obtained from the antibody-producing cells of the experimental animals by cell fusion in the known manner. In the same way, human monoclonal antibodies can be produced according to known methods. Antibodies of this type could then be used both for diagnostic tests, in particular of endometriotic cell tissue, or else for the therapy.

For example, samples such as body fluids, in particular human body fluids (e.g. blood, lymph or CSF) may be tested with the aid of the ELISA technique on the one hand for the presence of a polypeptide encoded by the endometriosis gene, on the other hand for the presence of autoantibodies against such a polypeptide. Polypeptides encoded by the endometriosis gene or fragments thereof can then be detected in such samples with the aid of a specific antibody, for example of an antibody according to the invention. For detecting autoantibodies it is preferably possible to employ recombinant fusion proteins which contain a part or a domain or even the complete polypeptide encoded by the endometriosis gene and which are fused to a protein domain which facilitates detection, for example maltose-binding protein (MBP).

Diagnostic tests may also be carried out with the aid of specific nucleic acid probes for detecting at the nucleic acid level, for example at the gene or transcript level.

Provision of the nucleotide and amino acid sequences and antibodies according to the invention further facilitates a targeted search for effectors of the polypeptides/proteins. Effectors are agents which act in an inhibitory or activating manner on the polypeptide according to the invention and which are capable of selectively influencing cell functions controlled by the polypeptides. These may then be employed in the therapy of appropriate pathologies, such as those based on invasive processes. The invention therefore also relates to a method for identifying effectors of endometriotic proteins where cells expressing the protein are brought into contact with various potential effector substances, for example low molecular weight agents, and the cells are analyzed for modifications, for example cell-activating, cell-inhibiting, cell-proliferative and/or cell-genetic modifications. In this way it is also possible to identify binding targets of endometriotic proteins.

Since many neoplastic diseases are accompanied by invasive processes, the discovery of the gene according to the invention additionally provides possibilities for the diagnosis, prevention and therapy of cancerous diseases.

The discovery of a gene involved in the responsibility for invasive processes not only opens up possibilities for the treatment of diseases based on cellular modifications of this type, but the sequences according to the invention may also be used in order to make such processes usable. This can be of importance, for example, for the implantation of embryos.

The present invention therefore also relates to a pharmaceutical composition which includes as active components nucleic acids, vectors, cells, polypeptides, peptides and/or antibodies, as mentioned before.

The pharmaceutical composition according to the invention may further contain pharmaceutically conventional carriers, excipients and/or additives and, where appropriate, further active components. The pharmaceutical composition may be employed in particular for the diagnosis, therapy or prevention of diseases associated with invasive processes. Furthermore the composition according to the invention may also be employed for the diagnosing a predisposition for such diseases, in particular for diagnosing an endometriosis risk.

The invention is illustrated in more detail by the following figures, sequence listings and examples.

### BRIEF DESCRIPTION OF THE DRAWINGS

FIG. 1. Shows a diagrammatic representation of the cDNA of the endometriosis-associated gene where only exons E1 or E5 are shown.

FIG. 2. (A) Diagram depicting DDRT-PCR performed with invasive and non-invasive passages of the endometriotic cell line EEC145T, leading to the identification of frag-1 mRNA. (B) The 391 bp cDNA was used as a probe to test for the presence of frag-1 mRNA in endometriotic and carcinoma cell lines. Poly A⁺RNA was prepared from the cell lines EJ28 (invasive bladder carcinoma), RT112 (non-invasive bladder carcinoma), EEC145T (p17=invasive passage 17; p33=non-invasive passage 33 of the endometriotic cell line) and Per143T (peritoneal cells immortalised with SV40T antigen). A Northern blot probed with ³²P-labelled frag-1 probe detected an mRNA of about 4 kb in the invasive endometriotic cell line. Lower panel: the membrane was reprobed with cytochrome C oxidase to check the integrity and loading of the RNA samples.

FIG. 3. (A) The complete 411 amino acid sequence of the frag-1 protein. The putative signal peptide is depicted in bold letters and the transmembrane domain is underlined. (B) Lanes 1 and 2 show the endogenous expression of frag-1 protein in pancreas and uterus sections, respectively, as detected by immunoblotting using the monoclonal antibody against frag-1; lanes 3 and 4 show the autoradiography of in vitro translated luciferase control cDNA and frag-1-BP, respectively after separation by SDS-PAGE; lane 5 depicts frag-1-GFP expressed in MCF-7 cells, as detected by monoclonal GFP antibody, lane 6 shows frag-1-GFP detected by the polyclonal antibody and lane 7 shows frag-1-GFP detected by the monoclonal antibody generated against frag-1.

FIG. 4. Membrane localization of frag-1. Frag-1 tagged with GFP (frag-1-GFP) or BP (frag-1-BP) was expressed in the eukaryotic epithelial cells: 12Z (human invasive endometriotic cell line), RT112 (human bladder carcinoma cell line, non-invasive), EJ28 (human bladder carcinoma cell line, invasive) and MCF7 (human breast carcinoma cell line, non-invasive). A-D show frag-1-GFP fluorescence and E-H show immunofluorescence signals using a mouse monoclonal antibody against the BP tag visualized by a mouse-specific fluorochrome-conjugated secondary antibody. The arrows indicate the expression of frag-1 at the membrane.

FIG. 5. Cell surface biotinylation of MCF7 cells transfected with frag-1-GFP. The biotinylated cell surface proteins were pulled down with neutravidin-coupled beads. The proteins present in various cell extract fractions were analysed by Western blots. (A) Frag-1-GFP was detected by anti-GFP antibody (lanes 1-5). (B) E-cadherin, a positive control membrane protein, was detected by a monoclonal antibody against E-cadherin (lanes 1-4) and (C) Pyruvate kinase, a negative control cytosolic protein, was detected with a specific antibody (lanes 1-4). UCX: untransfected cell extract, CX: transfected cell extract, sup: supernatant after pull-down of the biotinylated fraction, BF: pulled down biotinylated fraction, C: control of neutravidin beads bound to non-biotinylated cell extract.

FIG. 6. Carboxyl-terminus of frag-1 is cytoplasmic. Frag-1-GFP transfected MCF7 cells were permeabilised (A and B) or not permeabilised (C and D), and then subjected to immunofluorescence staining with anti-GFP antibody and Alexa 594-labelled secondary goat anti-mouse antibody (B and D: red fluorescence). Intrinsic GFP fluorescence is green (A and C). Frag-1-GFP could be detected in permeabilised cells by immunostaining with anti-GFP antibody (B) but not if the cells were not pemeabilised (D).

FIG. 7. Colocalization of frag-1-GFP with endogenous E-cadherin at the membrane in MDCK cells (A-C); and in MCF7 cells (D) along the xy-axis as seen in the confocal microscope. Colocalization at the junctions is seen along the xz-axis with the confocal microscope (E, F).

FIG. 8. Interaction between frag-1 and E-cadherin shown by coimmunoprecipitation. (A) MCF7 cells transfected with frag-1-GFP (lanes 1, 3) or with GFP (lanes 2, 4) were subjected to immunoprecipitation with anti-GFP. First, 10% of the total cell extract (Input) was immunoblotted (IB) with anti-GFP (upper panel) and anti-E-cadherin plus anti-β-catenin (middle panel) antibodies. Coimmunoprecipitations (Co-IP) were performed with anti-GFP antibody and the immunoprecipitates subjected to immunoblotting with anti-E-cadherin then anti-β-catenin antibodies (lanes 3, 4). (B) In the reverse experiment, the cell extracts from MCF7 cells transfected with GFP (lanes 1, 3) or frag-1-GFP (lanes 2, 4) were subjected to immunoprecipitation (IP) with anti-E-cadherin antibody. Input panels depict 10% of the cell extracts immunoblotted with anti-GFP antibody (upper panel), or endogenous E-cadherin protein immunoblotted with anti-E-cadherin antibody (lower panel). Coimmunoprecipitations (Co-IP) were performed with E-cadherin antibody, and frag-1 was detected by immunoblotting with anti-GFP antibody as seen in lane 4. CX denotes the total cell extract. (C) Coimmunoprecipitation of N-cadherin and frag-1-GFP. A: EJ28 cells were transfected with GFP (lanes 1, 3) or frag-1-GFP (lanes 2, 4). Input shows 10% of the total cell extracts (lanes 1, 2). Immunoprecipitation (Co-IP) was performed with GFP-antibody (lanes 3, 4). Immunoblotting was performed with antibodies against GFP, N-cadherin and β-catenin. No interaction of frag-1 with N-cadherin and β-catenin was observed. (D) Direct interaction of β-catenin with the cytoplasmic domain of frag-1 (GST-CPD-frag) in an in vitro pull-down assay. Full-length β-catenin was translated in vitro using ³⁵S methionine. GST and GST-CPD-frag were purified on glutathione sepharose beads, then incubated at RT for 1 h with radioactively labelled β-catenin. After washing the beads, samples were prepared and subjected to SDS-PAGE and autoradiography. Lane 1: radioactive β-catenin as input, lane 2: the marker, lane 3: GST alone with β-catenin and lane 4: GST-CPD-frag with β-catenin. FIG. 9. Effect of scatter factor (SF) on MDCK cells. 6 h after addition of SF (20 ng/ml) to the cells, cell-cell contacts were disrupted but colocalization of frag-1-GFP (green) and endogenous E-cadherin (red) could also be seen intracellularly. (A) MDCK cells transfected with frag-1-GFP before SF/HGF treatment; (B) cells after SF/HGF treatment.

FIG. 10. pTOPFLASH assay with frag-1 stable cell line. pTOP and pFOP plasmids were transfected into GFP and frag-GFP stable cell lines and the activity of the luciferase reporter was measured. pTOP activity was approximately 150-fold higher in the frag-1-GFP stable cell line compared to the GFP stable cell line. Luciferase activity is measured as relative light units (RLU).

SEQ ID NO. 1 represents a nucleotide sequence which contains genetic information coding for the endometriosis-associated gene, where an open reading frame extends from nucleotide 3 to 911, and

SEQ ID NO. 2 represents the amino acid sequence of the open reading frame of the nucleotide sequence shown in SEQ ID NO. 1, where the amino acid sequence of the open reading frame extends from amino acid 1 to 302.

SEQ ID NO. 3 represents a nucleotide sequence like that of SEQ ID NO. 1 but it contains an additional 84 nucleotides at the 5′ end, the open reading frame extends from nucleotide 3 to 995.

SEQ ID NO. 4 represents the amino acid sequence of the open reading frame of the nucleotide sequence shown in SEQ ID NO. 3, where this amino acid sequence has 320 amino acids of which the C-terminal 302 are identical to those in SEQ ID NO. 2.

SEQ ID NO. 5 represents of the nucleotide sequence of the possibly present additional exon 4a consisting of the 218 nt shown, where exon 4a, if it is present, is located between nucleotide 1054 and 1055 (in relation to SEQ ID NO. 3).

### EXAMPLES

### Example 1

### Identification of the Endometriosis-associated Gene called Frag-1

### Example 1.1

### Cell Culturing

To identify an endometriosis-associated gene, invasive and noninvasive cells of the epithelial endometriotic cell line EEC145T⁺ were used. The cells were cultured in Dulbecco's medium (DMEM) with 10% fetal calf serum and diluted 1 : 5 2× per week (passage). For comparison of the expression patterns by means of DDRT-PCR (see below) invasive cells of passage 17 and noninvasive cells of passage 33 were used. The cells were transformed with SV40 and analyzed by differential display reverse transcription polymerase chain reaction (DDRT-PCR).

### Example 1.2

### DDRT-PCR

This method developed by Liang and Pardee is a method for distinguishing expression patterns of different cell types or the alteration in the expression pattern of one cell type under different living conditions or during altering stages of development (Liang and Pardee (1992), Science 257, 967-971). The basis of the DDRT-PCR technique is based on the idea that in each cell about 15,000 genes are expressed and that in principle each individual mRNA molecule can be prepared by means of reverse transcription and amplification with random primers.

In this example the cellular polyA⁺ RNA was initially transcribed into cDNA with the aid of several different dT₁₁VX primers (downstream primers, anchor primers). The resulting cDNA populations were then PCR-amplified using 4 downstream and 20 upstream primers from the RNA Map™ Kit from Genhunter, Nashville (1994), with the addition of a radiolabeled nucleotide. After the amplification the reaction mixtures were concentrated in vacuo and the obtained cDNA fragments were fractionated in a six-percent native PAA (polyacrylamide) gel. DNA detection was carried out by autoradiography. PCR mixtures showing distinct differences in the band pattern for the two cell variants to be studied were repeated twice in order to test reproducibility. If the previously found differences were confirmed, the bands were eluted from the gel according to known methods, reamplified, cloned and sequenced.

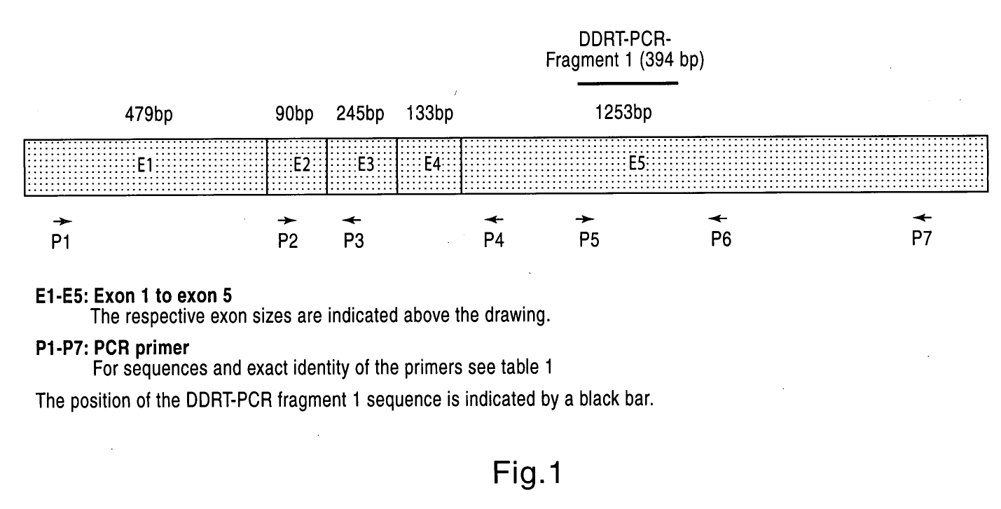

By this method a 394 bp fragment (fragment 1, nucleotides 1235 to 1628 of the nucleic acid sequence depicted in SEQ ID NO. 1, see also FIG. 1) was found which was specific for the invasive cell variant. This fragment 1 was used as a probe in Northern blot analysis (see below)

### Example 1.3

### Analysis of the Fragment 1 Expression Profile in Human Northern blot Analyses

To test the expression pattern for DDRT-PCR fragment 1, Northern blot analyses were carried out. For this 20 μg of total RNA or 4 μg of polyA+ RNA were fractionated in 1% denaturating agarose gels and transferred onto a nylon membrane overnight. The RNA was fixed to the membrane by irradiation with UV light. Hybridization with ³²P-labeled probes (labeling by means of RPL kit from Amersham) took place overnight in a formamide-containing hybridization solution at 42° C. Subsequently the membrane was washed under increasing stringency until the spots of radioactive emission were of measurable intensity. The hybridization pattern was visualized by putting on an X-ray film (NEF-NEN, DuPont) and exposing over several days. To determine the expression pattern for DDRT-PCR fragment 1, Northern blot analyses were carried out using RNA from the following cells or tissues:

invasive cells of the epithelial endometriotic cell line EEC145T⁺ (passage 17)

noninvasive cells of the epithelial endometriotic cell line EEC145T⁺ (passage 33)

cells of the peritoneal cell line EEC143T⁺

endometrial tissue

cells of the invasive human bladder carcinoma cell line EJ28

cells of the noninvasive human bladder carcinoma cell-line RT112

After hybridization with the probe for DDRT-PCR fragment 1 an mRNA of about 4 kb was detectable, and it was exclusively detectable in the invasive variant of the endometriotic cell line EEC145T⁺.

Further human tissues were tested. In the spleen an mRNA of 4 kb in length was found which hybridized unambiguously with fragment 1, and in brain mRNAs of 4 kb and >9 kb in length, respectively, were found.

Northern blot analyses were carried out according to the manufacturer's protocol using two human multiple tissue Northern (MTN) blots from Clontech. Expression was tested in the following tissues: colon, small intestine, heart, brain, testicles, liver, lung, spleen, kidney, ovaries, pancreas, peripheral blood leukocytes, placenta, prostate, skeletal muscle, thymus. The expression pattern obtained using the radiolabeled 3′ probe “DDRT-PCR fragment 1” appears as follows:

| | |
|---|---|
| 4 kb mRNA (expected size): | brain, |
| | spleen, |
| | pancreas |
| 9.5 kb mRNA: | brain |

In the remaining tissues no specific hybridization was detectable.

### In-situ Hybridization

To elucidate the cellular expression pattern, mRNA in-situ hybridizations were carried out on 10 μm paraffin sections of different tissues. For this the “DDRT-PCR fragment 1” was employed as digoxigenin-labeled RNA probe. The detection reaction was carried out by means of a digoxigenin-specific antibody coupled to alkaline phosphatase (A). BM Purple served as a substrate for AP and forms a blue precipitate after dephosphorylation. The results are listed in the following table and show predominant expression in invasive/migrating cells.

| | Weak, not quite |
|---|---|
| | unambiguous |
| Strong expression | expression |
| epithelial cells from endometriotic | skeletal muscle |
| lesions | heart |
| carcinomas | sarcomas |
| lymphatic infiltrates | |
| thymus | |
| germinal centers of lymph follicles | |
| (spleen) | |
| somewhat weaker: | |
| epithelial cells of the endometrium | |
| angiogenetic endothelial cells | |
| migrating nerve cells | |

### Example 1.4

### RT-PCR

RT-PCR (reverse transcription PCR) provides a sensitive method for testing the expression pattern.

For this, 1 μg of the appropriate polyA⁺ RNA was transcribed into cDNA with the aid of 400 U of M-MLV reverse transcriptase (Gibco-BRL) in a total volume of 30 μl. 1 μl of this was employed for the subsequent PCR with different primer combinations.

The PCR primers P1 to P7 used are depicted in table 1 (see FIG. 1).

TABLE 1

| | Sequence |
|---|---|
| | (nucleotide position in |
| Number | relation to SEQ ID NO. 1 |
| P1 | 5 -CCAGCTGCTGCCAAATCC-3 |
| | (36-53) |
| P2 | 5 -CATCATGGTCATAGCTGC-3 |
| | (545-562) |
| P3 | 5 -AGCGTCTCATCGGTGTAC-3 |
| | (793-776, reverse primer) |
| P4 | 5 -AACAGAAGTGGTAGGTGC-3 |
| | (1080-1063, reverse primer) |
| P5 | 5 -AAAGGGACGGGAGGAAGC-3 |
| | (1243-1260) |
| PG | 5 -CCAAAGTAGAAAACACTG-3 |
| | (1612-1595, reverse primer) |
| P7 | 5 -GCTTGTATGACACACACG-3 |
| | (2150-2133, reverse primer) |

RT-PCR experiments were carried out using polyA⁺ RNA from different cell lines and tissues and using different primer combinations. The results are depicted in table 2.

TABLE 2

| PC | P17 | P33 | Per | EM | EJ28 | RT112 | E | EE | PEE |
|---|---|---|---|---|---|---|---|---|---|
| P1 − | + | n.d. | n.d. | n.d. | n.d. | n.d. | n.d. | n.d. | n.d. |
| P4 | | | | | | | | | |
| P2 − | + | n.d. | n.d. | n.d. | n.d. | n.d. | n.d. | n.d. | n.d. |
| P6 | | | | | | | | | |
| P5 + | + | n.d. | n.d. | n.d. | n.d. | n.d. | n.d. | n.d. | n.d. |
| P7 | | | | | | | | | |
| P5 + | + | − | − | + | − | − | + | + | + |
| P6 | | | | | | | | | |
| P1 + | + | − | − | + | − | − | + | + | + |
| P3 | | | | | | | | | |

PC = primer combination
P17 = endometriotic cell line EEC145T, passage 17, invasive
P33 = endometriotic cell line EEC145T, passage 33, non invasive
Per = peritoneal cell line Per143T
EM = endometrial tissue
EJ28 = invasive bladder carcinoma cell line
RT112 = noninvasive bladder carcinoma cell line
E = endometrial tissue
EE = endometrial tissue of an endometriosis patient
PEE = peritoneal endometriosis biopsy
n.d. = not determined

The RT-PCR results confirmed the fragment 1-specific expression in the early passages (passage 17, passage 20) of the endometriotic cell line EEC145T⁺. As a deviation from the Northern blot analyses it was possible to show in addition a weak expression in the endometrium.

### RT-PCR Analyses Using Intron-bridging Primers

To test possible alternative exons, RT-PCR experiments using intron-bridging primers were carried out. In this connection it was possible to show at least one further mRNA species which exists alongside the mRNA described and which contains a further exon (4a) of 218 bp in length between the 4th and 5th exons. This exon is located in the 3′-UTR (untranslated region), that is to say after the coding region. The sequence of exon 4a is listed below.

gcggttgtcc ggaatgccag tggctcctgg gcagatgtgc
accccagatt cagcctttgt gatagattcc aacacgttct
ggcctcagac cacctttgtg gtggggccag actgctctgg
gcaaagtgaa gctggccttt atgctccaag gaagggggcc
tcgagagcag gcctgcattg gctctcggac taattcgcga
tcatctttca tacagcag

### Nucleotide Sequence of the Alternative Exon 4a

### Example 1.5

### Preparation of the cDNA Phage Bank EEC14

The cDNA phage bank EEC14 was prepared according to the method of Short, J. M. et al. (1988) Nucleic Acids Res. 16: 7583-7600.

Initially, reverse transcription of polyA⁺ RNA from invasive cells (passage 17) of the epithelial endometriotic cell line EEC145T⁺ was carried out. The primer used here consists of an XhoI cleavage site and a poly(dT) sequence of 18 nucleotides in length. An adapter including an EcoRI cleavage site was ligated to the cDNA fragments produced. The two restriction sites permit directed insertion of the cDNA fragments into the ZAP Express™ vector. Inserts can be excised from the phage in the form of a kanamycin-resistant pBK CMV phagemid.

### Example 1.6

### Phage Bank Screening

The DDRT-PCR fragment 1 (394 bp) was used as a probe in order to screen 10⁶ pfu (plaque forming units) of the cDNA phage bank EEC14 according to the manufacturer's protocol (Stratagene). Labeling of the probe with digoxigenin (Boehringer Mannheim) was carried out with the aid of PCR. The plaques formed after infection of the bacterial strain XL 1blue MRF′ were transferred onto a nylon membrane and hybridized thereon with the abovementioned probe. Detection of the hybridized, digoxigenin-labeled probe was carried out according to the chemiluminescence protocol by Boehringer Mannheim.

Positive plaques were selected and subjected to rescreening. The positive plaques from the rescreening were employed for the excision. Excising the vector portion from the phage by means of ExAssist helper phages resulted in kanamycin-resistant pBK CMV phagemids which could be isolated and sequenced after amplification in the bacterial strain XLOLR™. The isolated phagemid clone Q2A contained the longest insert of 2.3 kb in size whose sequence was determined and is shown SEQ ID NO. 1. The DDRT-PCR fragment 1 sequence is found as nucleotides 1235 to 1628 in relation to SEQ ID NO. 1.

### Example 1.7

### Southern Blot Analysis

10 μg of genomic DNA from female and male subjects were cleaved with various restriction endonucleases. The fragments were fractionated in an agarose gel and transferred onto a nylon membrane. Hybridization with the digoxigenin-labeled DDRT-PCR fragment 1 was carried out on this membrane.

Hybridization was detectable by chemiluminescence according to the Boehringer protocol. Using various restriction endonucleases only one band in each case was detected in both the female and male DNA samples. This result suggests that the gene on which fragment 1 is based is a single, non-sex-specific gene. Since then, two genomic clones PAC J1472 and PAC N1977 have been isolated using DDRT-PCR fragment 1.

### Example 1.8

### Fluorescence In Situ Hybridization (FISH)

The genomic clones obtained in Example 7 were localized on chromosome 1 (1p36) by means of fluorescence in situ hybridization (Lichter et al. (1990), Science 247:64-69).

### Example 1.9

### Production of Specific Antibodies

Nucleotides 584 to 909 of the abovementioned cDNA sequence were cloned by suitable restriction cleavage sites into the expression vector pMAL cRI. To express the sequence the construct was transformed into E. coli DH5 α cells. The translated protein fragment was cut out of an SDS polyacrylamide gel and employed for immunizing rabbits.

### Example 1.10

### RACE (Rapid Amplification of cDNA Ends)

Since the length of the cDNA clone Q2A (see Example 6) differs from the size of the detected mRNA (about 4 kb), RACE experiments were carried out to obtain further sequence information. With the aid of this method it is possible to obtain cDNA sequences from an mRNA template between a defined internal sequence and unknown sequences at the 5′ or 3′ end. The 3′ end of clone Q2A could be confirmed by 3′RACE experiments starting from the 5th exon.

For the 5′RACE, first strand synthesis of the cDNA was carried out using a gene-specific primer which hybridizes in the 1st exon, and then a homopolymeric nucleotide tail was attached with the aid of the enzyme terminal transferase. This attached sequence permitted amplification of the sequence region located between the gene-specific primer and the homopolymeric nucleotide tail. This made it possible to obtain the following additional sequence which is located 5′ from the Q2A sequence and belongs to the first exon:

| | |
|---|---|
| cc cgg ccg ccc cga gtg gag cgg atc cac ggg cag atg cag atg cct | 47 |
| Arg Pro Pro Arg Val Glu Arg Ile His Gly Gln Met Gln Net Pro | |
| 1 5 10 15 | |
| cga gcc aga cgg gcc cac agg ccc cgg gac cag gcg gcc gcc ctc gtg . . . | 95 |
| Arg Ala Arg Arg Ala His Arg Pro Arg Asp Gln Ala Ala Ala Leu Val . . . | |
| 20 25 30 | |

The underlined sequence represents the first nucleotides of the Q2A sequence, the sequence in front of it corresponds to the novel sequence obtained by 5′RACE. The open reading frame fits into the one already derived for fragment and contains two putative start codons (underlined).

The nucleotide sequence which has the sequence previously obtained and is depicted in SEQ ID NO. 1 and the additional 84 nt at the 5′ end is depicted in SEQ ID NO. 3.

### Example 1.11

### Cellular Localisation of the Frag-1 Protein

By means of computer-based analyses of the almost complete frag-1 cDNA an open reading frame could be detected coding for a protein having a total length of 411 amino acids. A further computer-based analysis of the amino acid sequence showed a significant outside→inside transmembrane domain within the protein, as well as a somewhat unusual signal peptide sequence comprising the amino acids 1-43. This fact renders it probable that frag-1 could be a transmembrane protein. The localisation of the frag-1 protein should, on the one hand, be performed by means of a birch profiline (BP)-tag and, on the other hand, as GFP (green fluorescent protein)-fusion protein. For this purpose the sequence coding for frag-1 was first cloned into a pcDNA3.1-vector (in-vitrogen, Leiden, Netherlands), which had already been furnished with the sequence of the birch profiline-tag. This frag-1-BP-vector was inserted into different eukaryotic cells by means of SuperFect (company Qiagen). About 40 h after transfection the cells were fixed with 4% paraformaldehyde, permeabilized with 0.2% of Triton X-100 and the frag-1 protein (frag-1 BP) tagged by the C-terminus was detected by means of a BP-specific antibody.

For the production of the frag-1-GFP fusion protein the commercially available vector pEGFP-N3 (Clontech, Heidelberg) was selected, which allows an expression of GFP at the C-terminus of frag-1. The complete coding sequence of frag-1 was also cloned into this vector, so that in the end a fusion protein develops consisting of the frag-1 protein having a length of 411 amino acids, at the C-terminus of which the GFP-protein is situated (frag-1-GFP). With the aid of this construct the expression was examined in the same eukaryotic cells as with the aid of the frag-1 protein tagged with BP. Approximately 40 h after SuperFect-transfection the cells were also fixed with 4% paraformaldehyde, washed with PBS and evaluated directly in the fluorescence-microscope. The preliminary result for the tested cell lines EEC145T⁺, 12Z (both epithelial endometriotic cell lines) and MCF-7 (mamma carcinoma-cells) can be described as follows:

### MCF-7

Those cells are mamma carcinoma-cells growing in typical epithelial cell associations due to their E-cadherin-expression and exhibiting the compact cell form characteristic of epithelial cells. Since these cells express frag-1 and, thus, possess the cellular background for a physiological frag-1 expression, and furthermore, rather possess epithelial cell character as compared to the endometriotic cell lines in culture, they were selected for first expression studies. In this context, it turned out that the expression patterns of the constructs explained above (frag-1-BP and frag-1-GFP) differ from one another. Whereas frag-1-BP for the most part gets stuck in the Golgi's apparatus, the frag-1-GFP also occurs in the cell membrane. The distribution into the two cell compartments, however, depends on the strength of expression of frag-1-GFP.

### EEC145T⁺

This cell line has already been described several times and served as starting point for the frag-1 isolation. For this reason it was interesting to examine the localisation in these E-cadherin-negative cells of epithelial origin. As compared to MCF-7 these cells do not exhibit the typical epithelial appearance, but rather possess a fibroblastoid growth behavior. In this respect, differences in the expression of both examined constructs, frag-1-BP and frag-1-GFR, can be noticed as well, membrane discolorations being again noticeable with both constructs. In this context, in cells expressing frag-1-BP a significant accumulation of the fusion protein in the Golgi's apparatus can be detected as well.

If frag-1 is actually a transmembrane protein which follows the typical synthesis route via endoplasmatic reticulum (ER) and Golgi's apparatus, an accumulation of over-expressed, not yet completely processed frag-1 protein in the Golgi's complex can easily be explained.

### 12Z

This cell line is also an epithelial endometriotic cell line, which was obtained by transfection of the SV40 T-antigen, and is, just like EEC145T⁺, E-cadherin-negative. These cells exhibit in culture a similar pattern of growth as EEC145T⁺, and, thus, were selected as second endometriotic cell culture system for controlling the frag-1 expression. The results of the frag-1-BP and frag-1-GFP expression obtained so far correspond to the results described above for the cell line EEC145T⁺.

### Example 1.12

### Expression Profile of Fragment-1-mRNA by Means of In Situ-hybridization

When preparing the expression profile of fragment-1-mRNA the method of in situ-hybridization was selected. This method renders possible to visualize the localisation of nucleic acids in tissues, cells and nuclei or chromosomes in vivo with the aid of labeled control probes. In this manner the spatial as well as the temporal expression pattern of various genes can be obtained and depicted. The advantage of this method, thus, consists in the detection of the mRNA to be found on the cellular level within a tissue association.

When determining the fragment-1 expression in various human tissue samples biochemically labeled RNA-probes (ribo probes) were used. The respective probe models were cloned within a vector having promoter sequences of bacteriophage-RNA-polymerases (e.g. Bluescript vectors by Stratagene with T3/T7-RNA-promoters). When producing the probes the probe models were linearized with a restriction endonuclease. Subsequent to the phenol/chloroform-extraction the sense- and antisense-ribo probes were produced by using the corresponding RNA-polymerases by means of in vitro-transcription, and thereby being marked with digoxigenin. In order to be able to hybridize the tissue samples with these produced ribo probes, the tissue first has to be freed from paraffin and to be hydrated in a declining ethanol series. Afterwards the preparations are pre-treated with several solutions and permeabilized thereby. Subsequently, the preparations are hybridized with the produced ribo probes overnight. For the immune-histochemical detection of the hybridized digoxigenin-labeled ribo probes anti-digoxigenin fab-fragments with conjugated alkaline phosphatase were employed. As substrate for this alkaline phosphatase BM Purple AP-substrate was employed resulting in a blue color-precipitate. The color reactions for each pair of probes (sense- and antisense-ribo probes) were always started simultaneously and stopped as soon as the blue coloring of the sense-ribo probe started.

By means of using different control probes the in situ-hybridization could be established and standardized during its course. Additionally, the hybridization results of these control probes furnished further information about the composition of the tissue. With the aid of a digoxigenin-labeled antisense-ribo probe of the DDRT-PCR-fragment-1 the various human tissue samples were examined as to their fragment-1 expression within the tissue association. In this connection a hybridization could be detected within the large intestine, embryo, endometrium (3 samples), endometriosis (3 samples), spleen, ovaries (2 samples) pancreas, placenta, prostate and thymus. Within these tissues the fragment-1-mRNA is primarily expressed within the epithelial cells, can, however, also be detected in migrating nerve cells, angiogenetic endothelial cells, lymphocytes as well as decidua and ovarian stromata. The increased fragment-1-mRNA expression in the endometriotic glands strikingly differs from the one in the endometrial glands. This increased expression can also be detected in carcinomas (10 samples) and sarcomas (3 samples). This increased expression is less detectable within the sarcomas. The sarcomas are malign soft-tissue tumors that are classified according to the departing mother tissue. Contrary thereto, a hybridization could not be detected within granular tissue, liver, lung and the thyroid gland.

### Example 2

### Characterisation of the Endometriosis-Associated Gene called Frag-1

### Example 2.1

### Identification of Frag-1 from Endometriotic Cell Line

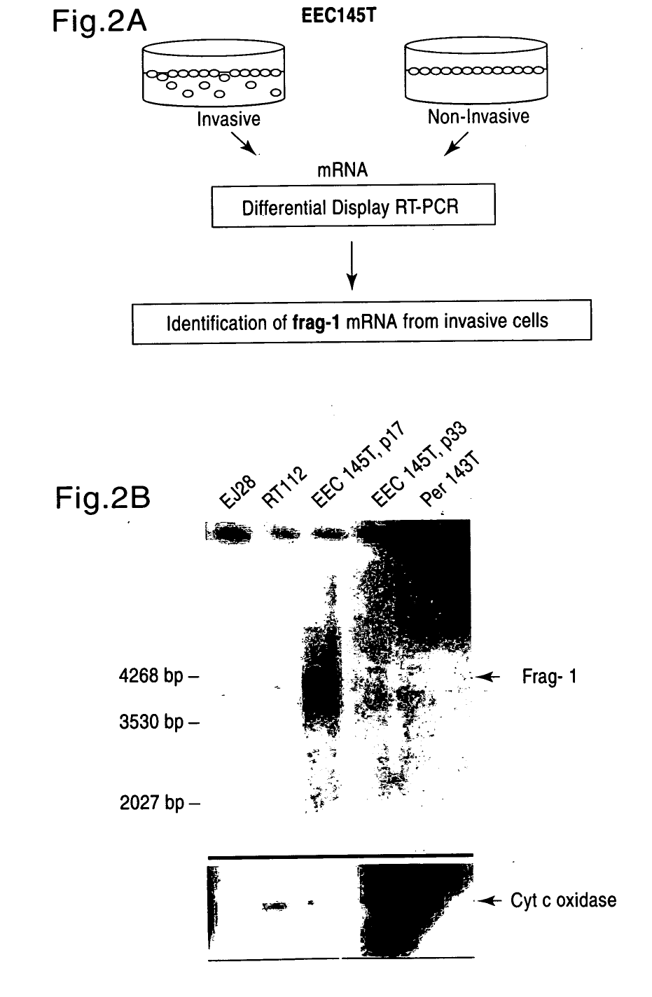

A cell line (EEC145T) from endometriosis lesions which has recently been established in the inventor's laboratory was found to be epithelial in nature (cytokeratin positive, E-cadherin negative). The fact that this cell line became non-invasive after a few passages prompted the inventors to use it as a tool for identifying markers differentially expressed during endometriosis (FIG. 2A). Therefore Differential Display Reverse Transcriptase PCR (DDRT-PCR) was performed with the invasive (p17) and non-invasive (p33) passages of this cell line.

DDRT-PCR was essentially performed as developed by (Liang and Pardee) using a commercially available kit (Genhunter Corporation, Nashville, USA). Briefly, the Genhunter kit contains four different downstream primers (T₁₂MA, T₁₂MG, T₁₂MT, T₁₂MC) used for first strand cDNA synthesis and twenty different upstream primers (AP-1 to AP-20) for amplification. The cDNAs amplified from poly A+ RNA from either invasive or non-invasive EEC145T cells in the presence of radioactively labelled nucleotides were separated on polyacrylamide gels, autoradiographed and the band patterns compared. Amplification products differentially and reproducibly found in either of the EEC145T variants (invasive or non-invasive) were cut out of the gel, re-amplified and cloned into a vector. The nucleotide sequences of the cloned products were determined and the differential expression pattern of the identified sequences validated by RT-PCR and Northern blots.

This reproducibly resulted in the isolation of a 391 bp DDRT-PCR fragment that was differentially expressed in the invasive EEC145T cell line. Northern blots (FIG. 2B) using the 391 bp fragment as a probe confirmed the presence of a corresponding message in invasive EEC 145T cells and revealed an mRNA of approximately 4 kb. The gene and its products (mRNA and protein) were called frag-1 (also called shrew-1).

### Example 2.2

### Isolation of the cDNA and Nucleotide Sequence Analysis

Screening of a ZAP Express™/EcoRI/XhoI custom cDNA phage library constructed from RNA of invasive passage p17 of EEC145T according to standard protocols led to the isolation of phagemid clone Q2A containing an insert of 2204 nucleotides including the original DDRT-PCR fragment.

Longer cDNA fragments could not be obtained from this library. Therefore, the rest of the cDNA was isolated by 5′ and 3′ RACE experiments. Therefore, the kit for RACE (Clontech, Germany) was used according to the manufacturer's instructions. Briefly, PCR was performed using Marathon ready cDNA from human brain, which is also positive for frag-1 and the anchor primer AP1 provided, which annealed specifically to the linker sequence on the cDNA. The sequence of the gene specific primer from within the known 391 bp sequence used for 3′RACE was 5′-gtgttggaagatgctacc-3′ and that of the primer used for 5′RACE was 5′-tgaactcagtctctgtgg-3′. To confirm the specificity of the product nested RACE was performed with a nested gene specific primer for 5′RACE: 5′-ggatttggcagcagctgg-3′ and a nested primer provided with the kit for 3′RACE: 5′-tagacggttggtgagtgg-3′.

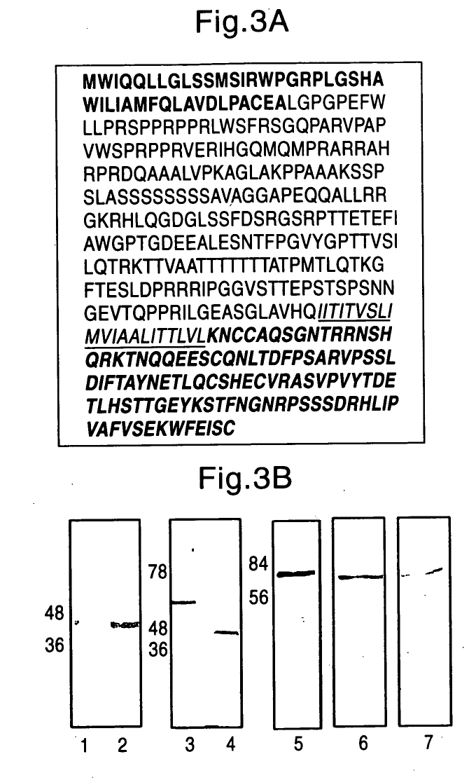

The cDNA finally obtained contained 2910 nucleotides and was identical to mRNA sequences in the EEC145T cells as revealed by overlapping RT-PCRs and DNA sequencing. It encodes a putative protein of 411 amino acids. The amino acid composition (FIG. 3) of frag-1 predicts a highly alkaline protein with an isoelectric point of 9.86 and a theoretical molecular mass of 44.5 kDa. A computer search for conserved protein motifs revealed a putative signal peptide of approximately 43 amino acids (bold in FIG. 3A), a putative transmembrane domain (underlined in FIG. 3A) and some potential sites for phosphorylation, glycolysation and myristylation.

### Example 2.3

### Expression of Frag-1 Protein

Two different types of antibodies were generated to analyse expression at the protein level. First, custom-made mouse monoclonal antibodies were produced against frag-1 in mice using the peptide sequence NH2-ACMTLQTKGFTESLDPRRRIPGGVS-amide by Nanotools, Teningen, Germany. The resulting monoclonal antibodies were tested against protein extracts from human pancreas and uterus in an immunoblot, since both these tissues were found to contain frag-1 mRNA as shown by Northern blot and RT-PCR analysis. As shown in FIG. 3B, lanes 1 and 2, both tissues were found to contain a protein of approximately 48 kDa corresponding to the predicted size of the frag-1 protein.

Secondly, polyclonal antibodies were generated in rats by genetic immunization against the putative cytoplasmic domain of frag-1. This antibody, however, only recognised the recombinant frag-1-GFP expressed in MCF7 cells, and not the endogenous protein.

For ectopic expression, frag-1 was cloned into two different expression vectors fused to either a 10 amino acid long birch profilin tag (frag-1-BP) or a green fluorescent protein tag (frag-1-GFP). Frag-1 cDNA isolated from the epithelial endometriotic cell line EEC145T was therefore cloned into eukaryotic expression vectors pEGFP-N3 (Clontech, Heidelberg, Germany) and into pcDNA3.1(+) with a BP tag using restriction sites introduced by PCR. PCRs were performed using Platinum Pfx-DNA polymerase (Invitrogen, Karlsruhe, Germany). The primers used for cloning into pEGFP-N3 contained the restriction sites BgIII and Acc651. The sequence of the forward primer was: 5′-agatctgaccatgtggattcaacagc-3′ and the reverse primer: 5′-ggtaccgcaggagatttcaaacc-3′. For cloning into the pcDNA 3.1(+) vector, the restriction sites HindIII and EcoRI were incorporated using the forward primer: 5′-aagcttgaccatgtggattcaacagc-3′ and the reverse primer: 5′-gaattccagcaggagatttcaaacc-3′.

To check whether these vectors expressed the predicted open reading frame protein of 411 amino acids, frag-1-BP was translated radioactively in vitro using a reticulocyte lysate kit. SDS-PAGE and autoradiography revealed that frag-1 could indeed be translated in vitro to produce a protein of approximately 48 kDa (FIG. 3, lane 4). The positive control used for in vitro translation was luciferase cDNA supplied by the manufacturer (FIG. 3, lane 3). Additionally, anti-GFP antibody detected a protein of the expected size of approximately 75 kDa in Western blots (FIG. 3, lane 5) in frag-1-GFP-expressing human epithelial MCF7 cells. Frag-1 polyclonal antibody raised in rats against the putative cytoplasmic polypeptide sequence gave a signal of comparable size in cell extracts of MCF7 transfected with frag-1-GFP (FIG. 3, lane 6). The frag-1 monoclonal antibodies that detected frag-1 in pancreas and uterus cell extracts (FIG. 3, lanes 1 and 2) also detected the recombinant transfected frag-1-GFP in MCF7 cell extracts (FIG. 3, lane 7). Thus, the predicted frag-1 amino acid sequence is translated in mammalian cells as confirmed by antibodies raised against putative ORFs.

### Example 2.4

### Membrane Localization and Orientation of Frag-1

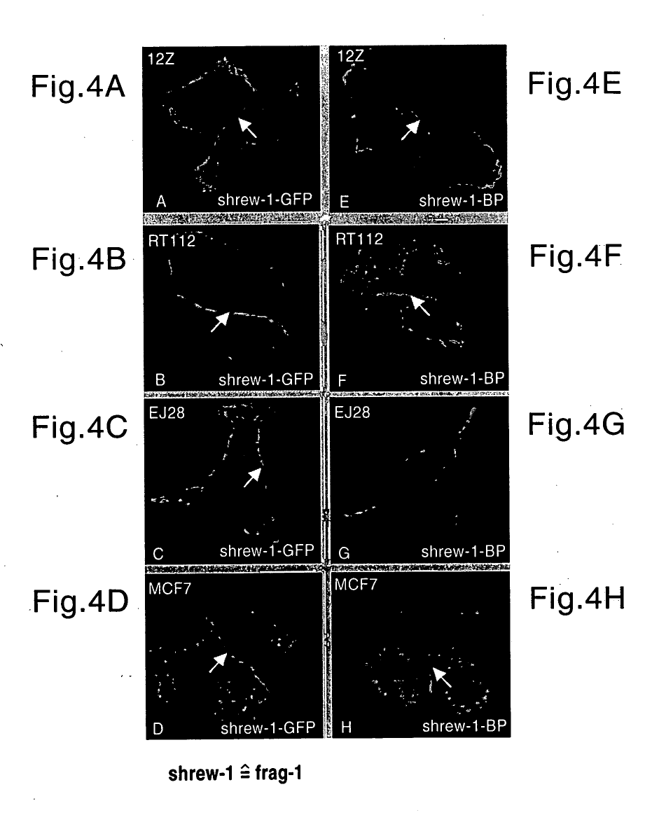

Frag-1 fused to two different tags (to rule out the possibility that cellular localization is affected by the tags) was used to determine the cellular localization of the protein. These studies were performed in epithelial cell lines 12Z, RT112, EJ28 and MCF7 transiently transfected with frag-1-GFP and frag-1-BP. In all cases, major pools of frag-1 appeared to be localized at the plasma membrane, especially at the regions of cell-cell contact, irrespective of whether RT-PCR showed that the cell lines contained endogenous frag-1, namely MCF7 and 12Z (FIG. 4; A, D, E, H) or not, i.e. RT112 and EJ28 (FIG. 4; B, C, F, G).

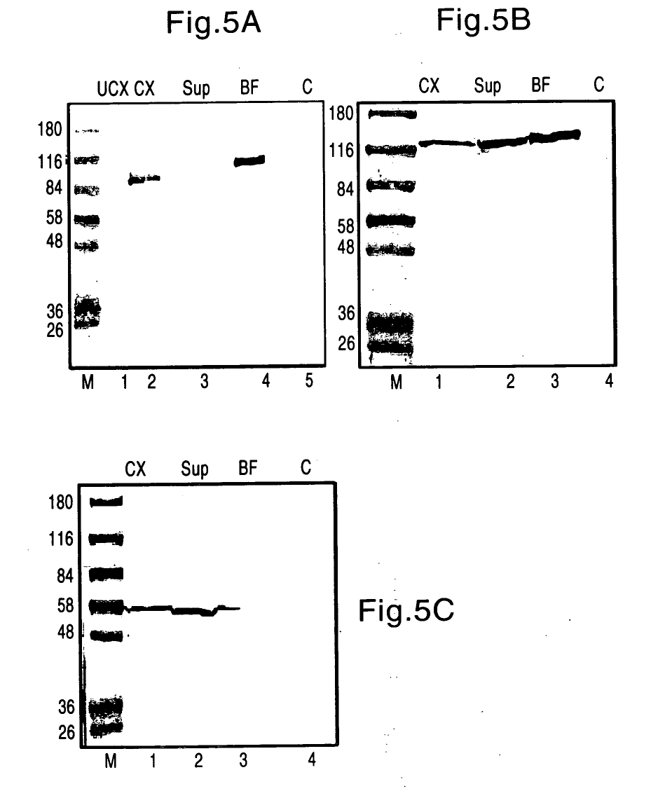

In order to check whether frag-1 is exposed on the cell surface, frag-1-GFP was transiently transfected into MCF7 cells. Surface-exposed proteins were then selectively biotinylated using a membrane-impermeable biotin. Therefore, the cell surface of confluent monolayers was labelled on ice with 0.5 μg/ml membrane-impermeable EZ-Link Sulfo-NHS-Biotin (Perbio, Bonn, Germany) in PBS, pH 9.0. After quenching (50 mM ammonium chloride in PBS, 0.1 mM CaCl₂, 1 mM MgCl₂) the cells were lysed in 0.5 ml of RIPA buffer (150 mM NaCl, 50 mM Tris pH 7.5, 0.25% sodium dodecyl sulphate, 0.1% Nonidet P-40) containing the protein inhibitor cocktail Complete (Roche, Germany) for 10 min at 4° C. Protein of each lysate was used for precipitation (16 h at 4° C.) with 30 μl of Neutravidin beads (Perbio, Germany). Immunoblotting using antibodies against GFP revealed that frag-1-GFP was present in the biotinylated protein fraction (FIG. 5A, lane 4) confirming that frag-1 is an integral component of the plasma membrane. E-cadherin, a transmembrane protein (FIG. 5B, lane 3) and pyruvate kinase, a cytosolic protein (FIG. 5C, lane 2) were used as positive and negative controls, respectively.

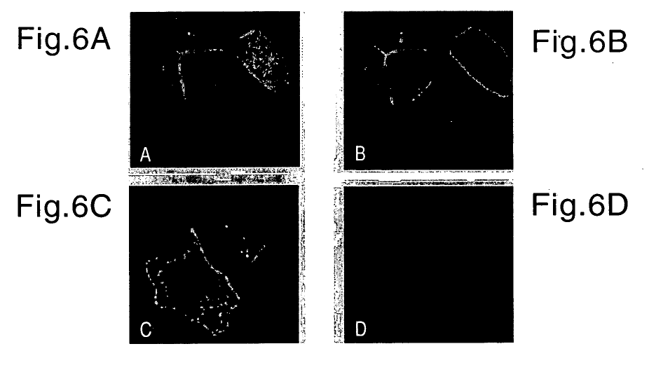

Furthermore, the carboxyl terminus of frag-1 was tested whether it is cytoplasmic by performing permeablization studies. MCF7 cells were transiently transfected with frag-1 tagged with a C-terminal GFP tag (frag-1-GFP). One aliquot of the transfected cells was permeabilized (FIG. 6; A, B) and immunodetection was performed using GFP antibody (FIG. 6; B, D) whereas the other aliquot was not permeabilized (FIG. 6; C, D) and immunostaining was performed on live cells using GFP antibody in the presence of sodium azide to prevent antibody-induced capping. The autofluorescence from frag-1-GFP (FIG. 6; A, C) could be seen in both cases, but antibody staining could only be seen with cells that were permeabilized. This clearly implies that the C-terminus is indeed cytoplasmic. A comparable result was obtained when a similar experiment was performed in MDCK cells.

### Example 2.5

### Colocalization of Frag-1 with E-cadherin at the Adherens Junctions

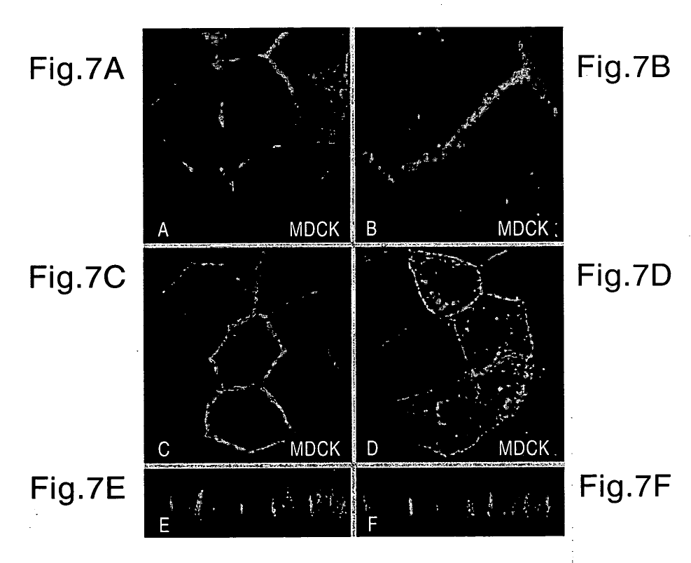

As seen in FIG. 4, frag-1-GFP was concentrated at sites of cell-cell contact. This was even more evident in epithelial cells that expressed E-cadherin at the membrane such as MCF7 (FIGS. 4 and 7) and MDCK cells (see also FIG. 7). Therefore, it may be possible that frag-1 and E-cadherin colocalise in these cells. Frag-l-GFP was transfected into MCF7 and MDCK cells and subsequently costained for endogenous E-cadherin by indirect immunofluorescence. Optical sectioning with confocal microscopy revealed that E-cadherin colocalises with frag-1-GFP along the xy-axis (FIG. 7; A-D). Interestingly, when the sections were recorded along the xz-axis (FIG. 7; E and F), frag-1 was found to colocalise with E-cadherin at the junctions.

Since E-cadherin is a marker of adherens junctions, it is presumable that frag-1 is also present in these junctions. Whether this colocalization was the result of frag-1 interacting specifically with E-cadherin or just a coincidence was further investigated.

### Example 2.6

### Interaction of Frag-1 with Cadherin-β-catenin Complexes in Polarised and Non-polarised Cells

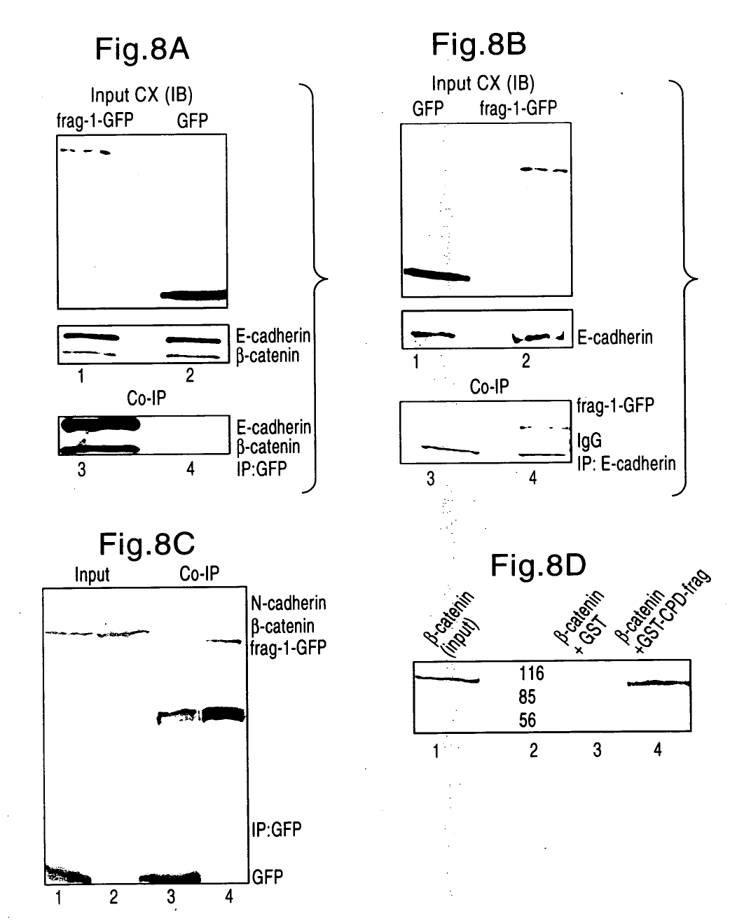

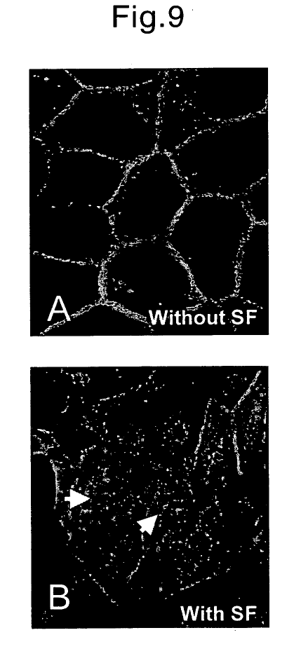

To check whether frag-1 can complex with E-cadherin, in vivo interaction assays were performed. MCF7 cells were transiently transfected with frag-1-GFP or the vector alone and grown to confluency. Cell extracts were prepared and transfection efficiencies were monitored by immunoblotting (IB) 10% of the total cell extract using GFP antibody (FIG. 8A; Input). The remaining cell extract was immunoprecipitated (IP) with GFP antibody and protein G-sepharose beads, then the whole complex was immunoblotted using E-cadherin antibody (FIG. 8A, lanes 3, 4). E-cadherin could be detected in the immunocomplex pulled down by monoclonal anti-GFP antibody. Complexing of frag-1 and E-cadherin could be observed in confluent but not in subconfluent cells. This suggested the presence of frag-1 in cadherin-catenin complexes only upon the formation of junctions. β-catenin was also detected on reprobing the same blot with the beta-catenin antibody (FIG. 8A). The reverse experiments also confirmed the same results when IP was done with E-cadherin antibody and frag-1-GFP could be detected in the same complex (FIG. 8B).

Furthermore, the ability of frag-1 to interact with cadherin in epithelial cell lines that are unable to form adherens junctions (for example EJ28 cells, an invasive human bladder carcinoma cell line expressing N-cadherin; FIG. 8C) was analyzed. EJ28 cells were transfected with frag-1-GFP and GFP alone. Monoclonal anti-GFP antibody was used for Co-IP assays. Therefore, cells were washed twice with ice cold PBS and lysed for 30 min at 4° C. in a buffer containing 10 mM Tris, pH 8.0, 150 mM NaCl, 5 mM EDTA, 1% Triton X-100, and 60 mM n-octyl-glucoside. Samples were precleared for 1 h at 4° C. using protein G-sepharose (20 μl, 1:1) and subjected to immunoprecipitation overnight at 4° C. using anti-GFP IgG (10 μl, mAb), anti-E-cadherin (5 μl, mAb 5H9), anti-Pan-cadherin (3 μl), followed by 2 h incubation with protein G-Sepharose (30 μl, slurry 1:1). After 4-5 washes with the immunoprecipitation buffer, samples were separated by SDS-PAGE (12% acrylamide) and transferred to nitrocellulose. Immunoblotting was performed according to standard protocols. For IB detection we used anti-N-cadherin, anti-β-catenin and anti-GFP antibodies. N-cadherin and β-catenin could not be detected in the immunocomplex pulled down by anti-GFP antibody. These data reiterate that frag-1 can interact with cadherin-catenin complexes in junctions of polarised epithelial cells but not with cadherin-catenin complexes (here N-cadherin) in non-polarised cells.

The results shown so far do not indicate whether the interaction between E-cadherin and frag-1 is due to direct binding of the proteins or is caused by an intermediate protein such as a scaffolding protein in the complex (β-catenin being a candidate). Therefore in vitro pull-down binding assays were performed between the cytoplasmic domain (CPD) of frag-1 (used as GST fusion protein) and in vitro translated β-catenin (FIG. 8D, lane 1) or full-length E-cadherin (not shown). For the in vitro pull-down binding assays β-catenin cloned in the expression vector pcDNA3.1 was synthesized by in vitro transcription-translation in the presence of ³⁵S-methionine using the TNT™-coupled reticulocyte lysate (Promega, Mannheim). Glutathione S-transferase cytoplasmic domain of frag-1 fusion (GST-CPD-frag) was expressed in E. coli BL21 pLysS. Pull-down assays were then performed according to standard protocols.

While E-cadherin could not be pulled down by GST-CPD-frag-1, β-catenin clearly interacted with the cytoplasmic domain of frag-1 (FIG. 8D, lane 4). Taken together, these data support that frag-1 interacts with β-catenin in adherens junctions. However, this does not exclude that frag-1 binds to other as yet unidentified components of the adherens junctions.

### Example 2.7

### Effect of Addition of SF/HGF on Colocalization of Frag-1 and E-cadherin

Cellular junctions were disrupted by adding scatter factor/hepatocyte growth factor (SF/HGF) to find out whether frag-1 and E-cadherin still colocalise after junction disruption. SF/HGF is a known cytokine that acts as a morphogen leading to epithelial-mesenchymal transitions. It is known to disrupt E-cadherin mediated junctions in MDCK cells through activation of its receptor c-met. During disruption, E-cadherin is transiently transported into recycling vesicles reported to contain caveolin-1. Addition of SF/HGF to MDCK cells transiently transfected with frag-1-GFP resulted in a dramatic change of the intracellular distribution of frag-1 (FIG. 9). Staining of the plasma membrane was reduced whereas intracellular particulate structures were labelled and also stained for E-cadherin. These results suggest that upon disruption of junctions by a physiological stimulus frag-1 is translocated together with E-cadherin to intracellular vesicles, further supporting the view that frag-1 is indeed complexing with E-cadherin.

### Example 2.8

### Effect of Stable Overexpression of Frag-1 on Transcriptional Activity of β-catenin

Since frag-1 can apparently participate in E-cadherin-mediated adherens junctions, the question was whether stable overexpression of frag-1 might affect cadherin-mediated junctions and consequently also the biochemical features of the cells. To answer this MDCK cells were transfected with frag-1-GFP or GFP alone as a vector control and selected with G418 to generate a cell line stably expressing frag-1. These cells showed fuzzy E-cadherin and β-catenin staining and had lost the regular honeycomb morphology characteristic of MDCK cells. This implied that the frag-1 stable cell line possibly no longer exhibits functional E-cadherin-mediated junctions, and that this might also influence the functional state of β-catenin.

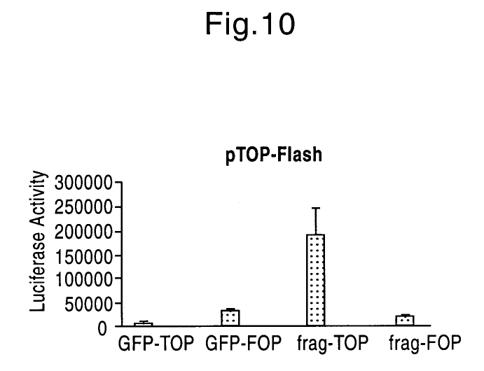

This question was particularly interesting since frag-1 is able to interact directly with β-catenin in vitro. Therefore it was tested whether transcriptional activation of β-catenin had emerged in the frag-1 stable cell line. A TOP-Flash assay was performed by transfecting the plasmids pTOP (containing synthetic Tcf/Lef-binding sites for testing β-catenin dependent transcription) and pFOP (negative control containing mutated binding sites), containing the luciferase reporter into the frag-1-GFP and vector cell lines. Therefore, MDCK cells stably expressing frag-1-GFP or GFP alone were seeded in 6-well plates at 300,000 cells/well. The following day, cells were transiently transfected using the Effectene? Transfection reagent (Qiagen, Hilden, Germany). For this, 0.4 μg of reporter gene plasmid DNA (TOPFLASH, FOPFLASH: Upstate Biotechnologies, Lake Placid, USA; UAS-5x-tk-Luc) was used. Each transfection was carried out in triplicate. Luciferase activities were measured 24 h after transfection using the Luciferase Assay System (Promega, Mannheim, Germany). Measured luciferase activity was as high as 150-fold in the stable frag-1-GFP cell line compared to the stable GFP cell line (FIG. 10). This could be interpreted as a consequence of the obvious disruption of the junctions, which could have led to the transcriptional activation of β-catenin by so far unknown mechanisms.

TABLE 3
Cell type-related expression chart of fragment-1

| epithelial cells | other cells |
|---|---|
| chorio-epithelium | decidua |
| large intestine cavities | germinal centers of the |
| embryonic epithelials | lymphatic follicles (spleen) |
| endometrial glands | lymphatic infiltrates |
| endometriotic glands | satellite cells (spleen) |
| endothelial cells, | nerv cells, migrating |
| angiogenetic carcinomas | ovarian stromata |
| pancreas glands | sarcomas |
| prostate glands | |
| tubal epithelium | |
| thymic epitheliocytes | |

As can be seen from these data, fragment-1 is mainly expressed in epithelial cells as well as in cells having an invasion or rather migration potential. Fragment-1 is particularly expressed in the carcinomatous areas of the liver and lung, although these tissues do not ordinarily express the fragment-1-mRNA. The liver contains the metastasis of a colonic carcinoma and the lung a papillary adeno-carcinoma.

1. A nucleic acid, comprising
(a) nucleotide sequences depicted in SEQ NO.1, 3 or/and 5, a combination or protein-encoding segment thereof,
(b) a nucleotide sequence corresponding to the sequence in (a) within the scope of the degeneracy of the genetic code or
(c) a nucleotide sequence hybridizing with the sequences in (a) and/or (b) under stringent conditions,
with the proviso that the nucleic acid is different from the sequences stated with accession numbers Z98886, Ac003017, Aa453993, AL023586 and Aa452856 in the EMBL EST database.

2. The nucleic acid according to claim 1, wherein it comprises
a protein-encoding segment of the nucleotide sequences depicted in SEQ ID NO. 3 or/and 5.

3. The nucleic acid according to claim 1, wherein it has a homology of more than 80% to the nucleotide sequences depicted in SEQ ID NO. 1, 3 or/and 5.

4. The nucleic acid according to claim 1, wherein
it codes for a polypeptide associated with invasive processes or for a segment thereof.

5. A modified nucleic acid or nucleic acid analog which
comprises a nucleotide sequence according to claim 1.

6. A polypeptide, encoded by a nucleic acid comprising
(a) nucleotide sequences depicted in SEQ NO.1, 3 or/and 5, a combination or protein-encoding segment thereof,
(b) a nucleotide sequence corresponding to the sequence in (a) within the scope of the degeneracy of the genetic code or
(c) a nucleotide sequence hybridizing with the sequences in (a) and/or (b) under stringent conditions.

7. The polypeptide according to claim 6, wherein said polypeptide has
(a) the amino acid sequence depicted in SEQ ID NO.2 or 4, or
(b) a homology of more than 70% to the amino acid sequence according to (a).

8. A modified polypeptide comprising an amino acid sequence according to claim 6.

9. A vector, comprising
at least one copy of a nucleic acid according to claim 1.

10. The vector according to claim 9, wherein said vector facilitates expression of the nucleic acid in a suitable host cell.

11. A cell transformed with a vector according to claim 9 or a nucleic acid comprising:
(i) nucleotide sequences depicted in SEQ NO.1, 3 or/and 5, a combination or protein-encoding segment thereof,
(ii) a nucleotide sequence corresponding to the sequence in (i) within the scope of the degeneracy of the genetic code or
(iii) a nucleotide sequence hybridizing with the sequences in (i) and/or (ii) under stringent conditions.

12. An antibody against a peptide according to claim 7 or a polypeptide encoded by a nucleic acid comprising
(a) nucleotide sequences depicted in SEQ NO.1, 3 or/and 5, a combination or protein-encoding segment thereof,
(b) a nucleotide sequence corresponding to the sequence in (a) within the scope of the degeneracy of the genetic code or
(c) a nucleotide sequence hybridizing with the sequences in (a) and/or (b) under stringent conditions.

13. An antibody according to claim 12, wherein said antibody is directed against a complete polypeptide or against a fragment thereof selected from a segment of amino acids 1 to 330 from SEQ ID NO.4.

14. A composition for pharmaceutical application, comprising at least one active component selected from the group consisting of:
(a) a nucleic acid comprising:
(i) nucleotide sequences depicted in SEQ NO.1, 3 or/and 5, a combination or protein-encoding segment thereof,
(ii) a nucleotide sequence corresponding to the sequence in (i) within the scope of the degeneracy of the genetic code or
(iii) a nucleotide sequence hybridizing with the sequences in (i) and/or (ii) under stringent conditions,
(b) a vector comprising at least one copy of a nucleic acid according to (a),
(c) a cell transformed with a nucleic acid according to (a) or with a vector according to (b),
(d) a polypeptide encoded by a nucleic acid according to (a),
(e) a peptide which has
(1) the amino acid sequence depicted in SEQ ID NO.2 or 4, or
(2) a homology of more than 70% to the amino acid sequence according to (1), and
(f) an antibody against a polypeptide according to (d) or against a peptide according to (e).

15. The composition according to claim 14, further comprising pharmaceutically conventional carriers, excipients and/or additives.

16. A method for producing antibodies comprising
obtaining a polypeptide encoded by a nucleic acid comprising:
(a) nucleotide sequences depicted in SEQ NO.1, 3 or/and 5, a combination or protein-encoding segment thereof,
(b) a nucleotide sequence corresponding to the sequence in (a) within the scope of the degeneracy of the genetic code or
(c) a nucleotide sequence hybridizing with the sequences in (a) and/or (b) under stringent conditions, and
administering said polypeptide or a fragment thereof as an immunogen to an animal, and
obtaining the resulting antibodies.
